(19) United States
(12) Patent Application Publication
(10) Pub. No.: US 2008/0075787 A1
(43) Pub. Date: Mar. 27, 2008

(54) Therapeutic Agent For Ophthalmic Diseases

(76) Inventor: Sawako Hibino, Hyogo (JP)

Correspondence Address: BIRCH STEWART KOLASCH & BIRCH, PO BOX 747, FALLS CHURCH, VA, 22040-0747, US

(21) Appl. No.: 11/791,530

(22) PCT Filed: Nov. 25, 2005
(86) PCT No.: PCT/JP05/22141

§ 371 Date: May 24, 2007

(30) Foreign Application Priority Data
Nov. 25, 2004 (JP) 2004-340203

(51) Int. Cl. A61K 35/50; A61P 27/02

(52) U.S. Cl. 424/583

(57) ABSTRACT

A therapeutic agent for ophthalmic diseases containing Laennec (trade name) as an active ingredient. Laennec, the active ingredient, exhibits a therapeutic effect on a wide variety of ophthalmic diseases by increasing tears and the like and is highly safe even though it is an animal-derived component. Therefore, the therapeutic agent is applicable to the prevention and/or treatment of various types of ophthalmic diseases, particularly corneal disorders, dry eye, asthenopia, inflammatorily ophthalmic diseases (e.g., meibomian gland dysfunction, Stevens-Johnson syndrome, Sjogren syndrome, uveitis) and ophthalmic diseases caused by active oxygen (e.g., cataract, glaucoma, age-related macular degeneration, optic disc atrophy).

### TECHNICAL FIELD

The invention relates to a therapeutic agent for ophthalmic diseases. More particularly, the invention relates to a therapeutic agent for ophthalmic diseases containing Laennec (trade name) as an active ingredient.

### BACKGROUND ART

Patients with ophthalmic diseases are increasing recently, such as dry eye and asthenopia due to wide use of television, personal computer, game machine and other digital appliances, and popularity contact lens. Subjective symptoms of dry eye and asthenopia include ocular dryness, irritation, itchiness, blurred vision and failure in adjustment in near or distant visual acuity, which are considered to be caused mainly by disorder of corneal epithelium due to abnormality of lacrimal fluid. Dry eye and asthenopia are diseases causing troubles in daily life, but radical treatment is not known yet. Various substances have been proposed as dry eye remedies (for example, Japanese Patent Application Laid-Open No. 9-194363), but most of them are synthetic products, and biological substances of high safety are being demanded.

Mainly steroids are used as remedy for inflammatorily ophthalmic diseases (for example, meibomian gland dysfunction, Stevens-Johnson syndrome, Sjogren syndrome, uveitis). Steroids are excellent in anti-inflammatory action, but have strong actions and hence severe side effects, and maximum caution is needed in administration, and they cannot be used easily or prescribed for a long period.

Other recent topics are troubles of organs and tissues by active oxygen, and opthalmologic field is no exception, and prevention and treatment of ophthalmic diseases caused by active oxygen (for example, cataract, glaucoma, age-related macular degeneration, optic disc atrophy) are demanded.

In the light of these problems, the inventor closely investigated into substance of high safety of biological nature, effective for prevention and treatment of ophthalmic diseases, and discovered that Laennec is effective and useful for prevention and treatment of various ophthalmic diseases, and completed the invention. That is, the invention presents a therapeutic agent for ophthalmic diseases containing Laennec as an active ingredient and effective for ophthalmic diseases in a wide range including dry eye and inflammatorily ophthalmic diseases.

Laennec is a medicine derived from placenta extract, and used in treatment of chronic hepatic diseases. Laennec was approved as medicine in 1974 in Japan, and is highly evaluated in safety. However, the action of Laennec on ophthalmic disease has not been known yet.

### DISCLOSURE OF THE INVENTION

The therapeutic agent for ophthalmic diseases of the invention contains Laennec as an active ingredient. Ophthalmic disease includes corneal disorder, dry eye, asthenopia, and inflammatorily ophthalmic disease (for example, meibomian gland dysfunction, Stevens-Johnson syndrome, Sjogren syndrome, uveitis), and ophthalmic diseases caused by active oxygen (for example, cataract, glaucoma, age-related macular degeneration, optic disc atrophy). The therapeutic agent for ophthalmic diseases of the invention is preferably used as oral dose or eye-drops.

### BRIEF DESCRIPTION OF THE DRAWINGS

FIG. 1 is a drawing showing an example of evaluation method of AD score of biological staining (fluorescein staining, rose-bengal staining).

FIG. 2 is a drawing showing changes in time of radius (r) of curvature of meniscus in Example 4.

FIG. 3 is a drawing showing changes in time of BUT (tear film breakup time) in Example 4.

FIG. 4 is a drawing showing changes in time of AD score in Example 4.

FIG. 5 is a drawing showing changes in time of value of Schirmer's test in Example 4.

### DETAILED DESCRIPTION OF THE PREFERRED EMBODIMENTS

The therapeutic agent for ophthalmic diseases of the invention contains Laennec as an active ingredient. Laennec injections are already available commercially, and Laennec preparations can be properly manufactured to be suited to the purpose of the invention.

The therapeutic agent for ophthalmic diseases of the invention is preferably administered orally, or used as eye-drops.

Oral preparations can be manufactured in proper dosage by mixing the Laennec injection or freeze-dried powder as required with pharmacologically acceptable additives (for example, carrier, vehicle, diluent), and examples of pharmaceutical preparation include tablet, powder, granule, and capsule.

Eye-drops are formulated by mixing Laennec with purified water, isotonic agent (for example, sodium chloride, glycerin), surface active agent (for example, polysorbate 80, polyoxyethylene alkyl ether), preservative (for example, sodium edetate, sodium sorbate), buffer agent (for example, sodium phosphate), pH regulator (for example, hydrochloric acid, sodium hydroxide), and other customary pharmaceutical components by ordinary methods. The liquid property is preferred to be near neutral pH (pH 5 to 8), and the osmotic pressure is also preferred to be near 1.

The content of Laennec in pharmaceutical preparation may be properly adjusted depending on the dosage form, disease to be treated, and patient's age, body weight, and symptoms.

The effective dose and schedule of administration of the medicine of the invention may be determined empirically, and are known to those skilled in the art. The dose is properly adjusted depending on the route, disease, and patient's age, body weight, and symptoms, and in case of eye-drops, the medicine of about 0.001 to 3% (w/v, same hereinafter), or preferably about 0.01 to 1% is dropped once to several times a day.

For internal use, the dose is selected in a range of 1 to 100 mg/kg body weight, preferably 2.5 to 50 mg/kg body weight, more preferably about 25 mg/kg body weight, which is divided in one to several portions a day.

The therapeutic agent for ophthalmic diseases of the invention can be applied in ophthalmic diseases in a wide range, and is particularly effective for prevention and treatment of corneal disorder, dry eye, asthenopia, and inflammatorily ophthalmic disease (for example, meibomian gland dysfunction, Stevens-Johnson syndrome, Sjogren syndrome, uveitis), and ophthalmic diseases caused by active oxygen (for example, cataract, glaucoma, age-related macular degeneration, optic disc atrophy).

### INDUSTRIAL APPLICABILITY

Laennec, the active ingredient contained in the therapeutic agent for ophthalmic diseases of the invention, is useful and effective for prevention and treatment of various ophthalmic diseases as shown in Examples below, and the therapeutic agent of the invention is useful for prevention and treatment of a wide range of ophthalmic diseases, and is very high in safety because the active ingredient is a biological substance.

### EXAMPLES

Examples of the invention are described below, but the invention is not limited to these Examples alone. In the following Examples, the pharmaceutical preparation and testing methods are as specified below.

(1) Pharmaceutical Preparation

Preparation of oral dose: Commercial Laennec injection (manufactured by Japan Bioproducts Ind. Co. Ltd.), 3 ampoules (6 ml), was freeze-dried, and freeze-dried powder was obtained and applied in a capsule, and a preparation containing 350 mg of the powder in capsule was manufactured and used.

Preparation of eye-drops: Commercial Laennec injection, one ampoule (2 ml), was mixed with about 8 ml of purified water, blended with preservatives or other additives as required, and dispensed in eye-drops containers disinfected by ethanol, and eye-drops preparations were manufactured and used. The pH of Laennec injection is 5.5 to 6.5, and the osmotic pressure ratio (ratio to normal saline) is about 1. Safety of the eye-drops preparation was confirmed by 28 adult healthy volunteers (16 men, 12 women, aged 24 to 68).

(2) Test Methods

(A) Biological Staining Test (Rose-Bengal Staining, Fluorescein Staining)

Using 2 μl of a mixed solution of 1% fluorescein and 1% rose-bengal, the cornea and conjunctiva were stained, and the stained diagram was recorded, and the degree of staining was recorded by scoring, and results were entered in the investigation sheet. Recording and scoring were evaluated objectively by using photographs as far as possible.

More specifically, the mixed solution of 1% fluorescein and 1% rose-bengal was dropped by 2 μl by using a micropipette with a disposable tip. Since the tip of the micropipette is disposable, it is low in risk of contamination, and a specific amount can be dropped, and the repeatability is high. Further, biological staining by two dye stuff and tear film breakup time (BUT) measurement can be done at the same time. Rose-bengal stains the epithelial cells having differentiation anomaly not covered with mucin on the cornea and conjunctiva. Fluorescein stains weak adhesion portion of epithelium of cornea and conjunctiva (barrier function broken portion) or epithelial defective portion, and is useful for observation of superficial punctate keratopathy by dry eye, corneal epithelial defect, or corneal or conjunctival ulcer. In fluorescein staining, the stained portion can be observed by slit-lamp microscope through cobalt-blue filter.

1) Fluorescein Staining, Rose-Bengal Staining

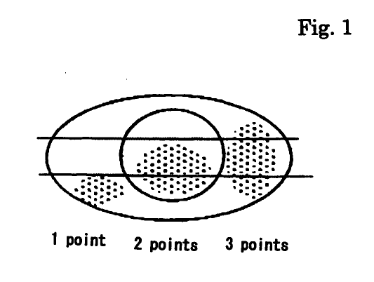

As shown in FIG. 1, in the corneal upper part, corneal middle part and corneal lower part, the rose-bengal staining degree was scored in three-point full marks, and the total points (AD score) were evaluated (nine-point full marks).

More specifically, epithelial disorder of cornea and conjunctiva by dry eye was evaluated often by the criteria proposed by van Bijsterveld (Diagnostic tests in the sicca syndrome, Arch. Opthalmol., 82: 10-14, 1969), which was known as AD score. In three events of ear-side conjunctiva, cornea, and nose-side conjunctiva, the staining degree was evaluated in three-point full marks, in a total of 9 points. Namely, no staining is 0 point, slight and partial staining is 1 point, medium staining of about ⅔ is 2 points, and heavy and full staining is 3 points. Examples are shown in FIG. 1.

2) Tear Film Breakup Time (BUT)

After staining by using 2 μl of the mixed solution of 1% fluorescein and 1% rose-bengal, the tear film was observed by slit-lamp microscope. Thickness of tear film reaches the maximum right after blinking, the tear fluid flows downward, and the thickness on the cornea decreases gradually. This is intended to measure the time until the tear film covering the ocular surface dried. Normally it is 10 seconds or more, and dry eye is suspected if shorter than 5 seconds.

(B) Conjunctival Hyperemia

It was evaluated in the following score.

0: no congestion, 1: slight congestion, 2: clear congestion, 3: significant congestion

(C) Schirmer's Test (First Method)

Wattman No. 1 filer paper (35 mm×5 mm) was used as Schirmer's paper. At inferior tarsal ear side, Schirmer's paper was applied for measurement of tear secretion during 5 minutes of natural blinking. The length of tear soaking in 5 minutes was measured. Normal value is 10 mm or more.

(D) Tear Meniscus Test (Meniscometry)

Tear meniscus is a tear stagnant position spreading like a band along the tarsal margin, the stagnant amount of tear is said to occupy about 70 to 95% of the entire ocular surface. In tear meniscus, the tear quantity was measured by meniscometry (Yokoi N. et al., Br. J. Opthalmol., 83: 92-97, 1999). In meniscometry, since tear meniscus is a concave plane, assuming a concave mirror, by projecting a target of horizontal lattice fringe, the mirror reflection image is analyzed by optical format, and radius (r) of curvature of tear fluid meniscus is measured without making contact.

As qualitative evaluation of tear volume or flow, various methods are known clinically, but the evaluation of tear meniscus height is highest in repeatability and is considered to be most useful for screening of dry eyes.

### Example 1

In 13 patients having subjective symptoms, for example, no lacrimation to external stimulation, irritation, fatigued condition, repeated eye rash, itchiness of eye, excess discharge of fat in eye, eye pain and urtication, upon obtaining their consent, 2 capsules of the Laennec preparation were orally administered after dinner every day (Laennec group). In 3 patients having similar subjective symptoms, placebo was administered (placebo group). Oral administration continued for 28 consecutive days.

Results are shown in Table 1.

TABLE 1

| | Placebo group | Laennec group |
|---|---|---|
| Meibomian | No change: 2 | Remarkably improved: 9 |
| gland dysfunction | None from | Improved: 3 |
| | beginning: 1 | None from beginning: 1 |
| Corneal epithelial | No change: 3 | Remarkably improved |
| disorder (corneal & | | (completely eliminated): 12 |
| conjunctival disorder) | | Improved: 1 |
| Dry eye subjective | No change: 3 | Improved: 12 |
| symptoms | | No change: 1 |
| Schirmer's test | No change: 3 | Improved (increased): 11 |
| (as index of tear volume) | | Aggravated (decreased): 2 |
| BUT (tear film breakup | No change: 3 | Improved (extended): 11 |
| time) | | No change: 1 |
| | | Aggravated (shortened): 1 |

Note 1)
Corneal epithelial disorder was evaluated by fluorescein and rose-bengal staining, BUT and conjunctival hyperemia.
Note 2)
“None from beginning” means there was no sign of meibomian gland dysfunction from the beginning.

As shown in Table 1, the oral preparation of Laennec acted locally, and was effective for treatment of various ophthalmic diseases. In particular, it was effective as remedy for meibomian gland dysfunction and dry eye. Especially, a notable effect was recognized in increase of tear volume. Subjective symptoms were improved in all patients in Laennec group. Throughout the test, no side effect was found in Laennec group.

In one case showing nerve drusen known as an initial sign of age-related macular degeneration, the drusen disappeared. In other case, optic disc atrophy was noted, but it tended to be improved. In other words, regeneration of optic nerve was suggested (improvement of optic disc recess ratio).

### Example 2

In 3 patients having similar subjective symptoms as in Example 1, after obtaining consent, the eye drops preparations of Laennec were administered, one drop each, 4 times a day at intervals of 3 to 4 hours. The term of treatment was 8 weeks, and the results were investigated every 2 weeks (by cornea staining and others).

As a result, subjective symptoms were improved in all 3 patients. In Schirmer's test (tear fluid dynamic), symptoms were improved in 2 cases and unchanged in 1 case. Tear meniscus (tear volume) was improved in all 3 patients. State of corneal and conjunctival epithelium was judged in 3 items (fluorescein and rose-bengal staining, BUT and conjunctival hyperemia), and was improved in all 3 cases.

As a result, eye drops preparations of Laennec are effective for various ophthalmic diseases, and are particularly effective for dry eye. No side effect was noted throughout the test.

### Example 3

Thioredoxin (TRX) naturally existing in the human body functions to protect the human cells and tissues from active oxygen inducing various diseases, and the stress on the human body can be estimated by measuring TRX concentration in the body. It is estimated that TRX concentration is high in a state of strong inflammation.

By obtaining the consent of 3 patients, tear fluid of patients was sampled before and after dropping of the eye drops preparation of Laennec, and the concentration of TRX contained in tear fluid was measured by using a commercial ELISA kit (only the right eye was tested in patient 3).

Results are shown in Table 2.

TABLE 2

TRX concentration (ng/ml)

| | | Before dropping | After dropping |
|---|---|---|---|
| Patient 1 | Right eye | 1098.7 | 74.4 |
| | Left eye | 9375.2 | 55.4 |
| Patient 2 | Right eye | 1403.0 | 511.6 |
| | Left eye | 4218.8 | 2078.2 |
| Patient 3 | Right eye | 8390.7 | 2434.1 |

As shown in Table 2, by dropping the eye drops preparation of Laennec, TRX concentration dropped dramatically, and it is confirmed that Laennec has an antioxidative activity like TRX, soothes inflammation, and reduces the stress on the eye.

### Example 4

In patients with severe dry eye (3 cases, 6 eyes) not improved sufficiently in symptoms by general eye drops, after obtaining prior consent, the eye drops preparation of Laennec was dropped four times a day. The underlying diseases of these patients were Sjogren syndrome, Stevens-Johnson syndrome and dry eye.

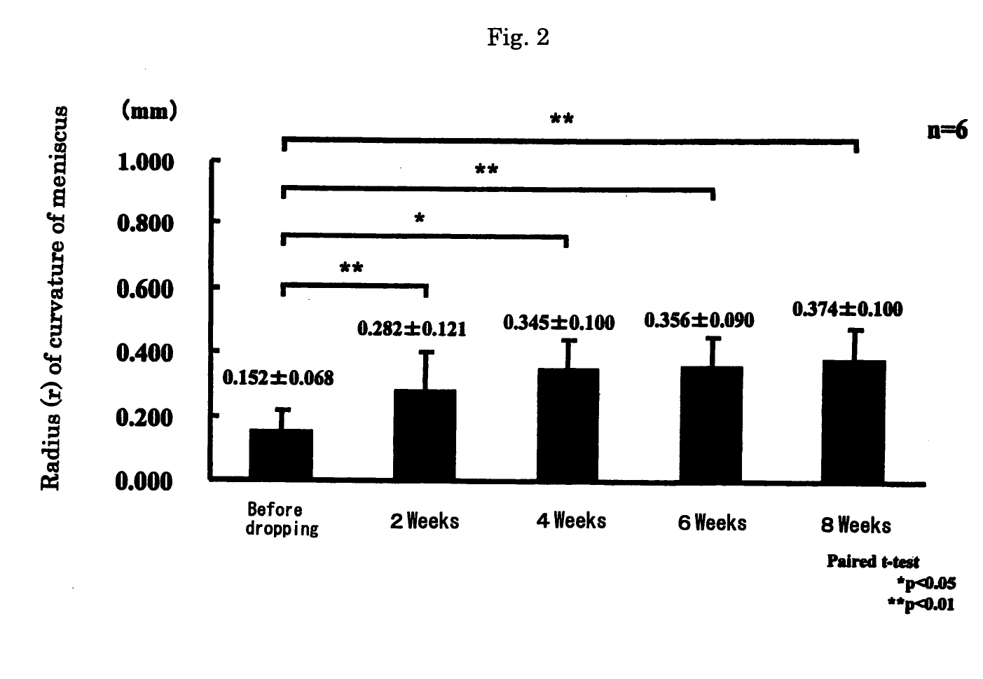

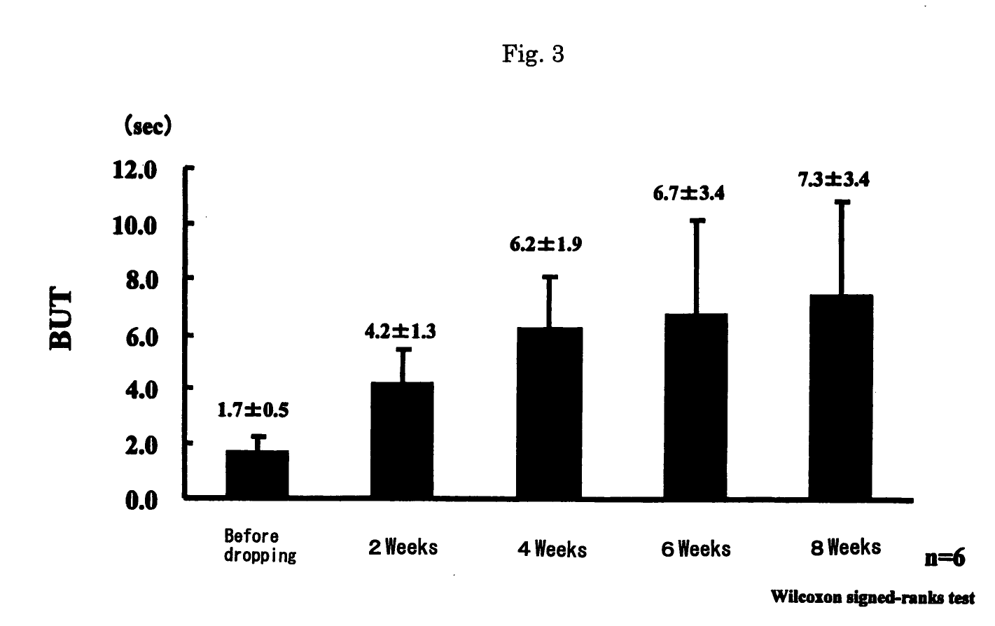

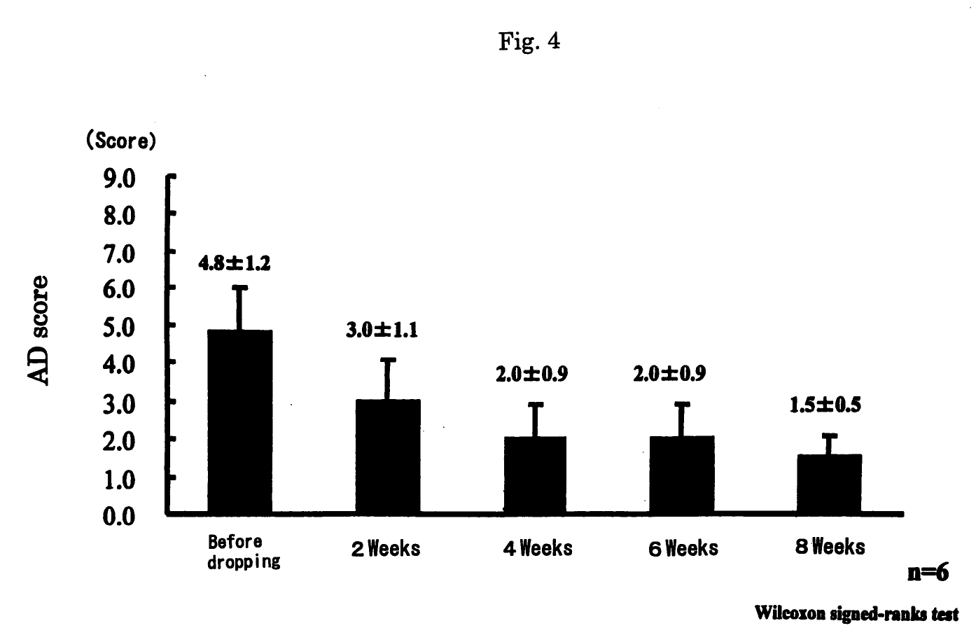

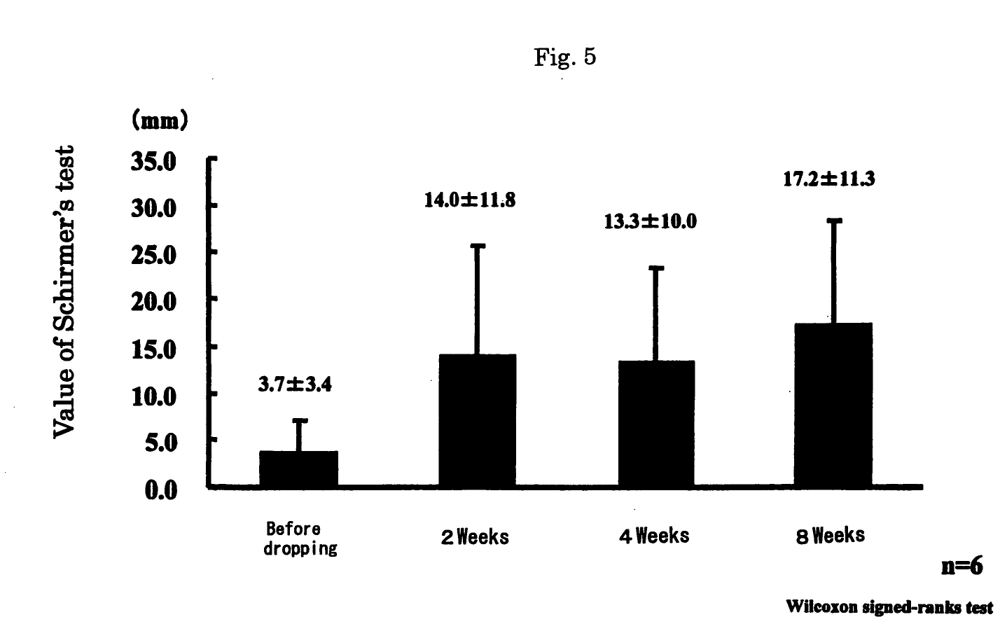

Before dropping, and in 8 weeks of dropping in every 2 weeks, the patients were tested by tear meniscus test, measurement of tear film breakup time (BUT), biological staining (fluorescein staining, rose-bengal staining) and Schirmer's test, and the radius (r) of curvature of meniscus, BUT, AD score, and value of Schirmer's test were measured. Average values of 3 cases (6 eyes) are shown in FIG. 2, FIG. 3, FIG. 4 and FIG. 5.

As shown in FIG. 2 to FIG. 5, the symptoms were improved. In particular, AD score was remarkably decreased, and the epithelial disorder was dramatically improved. Therefore, the eye drops preparation of Laennec is confirmed to have effects of increasing the tear fluid, and stabilizing the ocular surface.

1. A therapeutic agent for ophthalmic diseases containing Laennec as an active ingredient.

2. The therapeutic agent of claim 1, wherein the ophthalmic disease includes corneal disorder, dry eye, inflammatorily ophthalmic disease and ophthalmic disease caused by active oxygen.

3. The therapeutic agent of claim 1 or 2, wherein the dosage form is oral preparation or eye drops.

4. A treating method of ophthalmic disease characterized by administering an effective amount of Laennec.

5. The treating method of claim 4, wherein the ophthalmic disease includes corneal disorder, dry eye, inflammatorily ophthalmic disease and ophthalmic disease caused by active oxygen.

6. A use of Laennec for manufacturing a therapeutic agent for ophthalmic diseases.
